(19) United States
(12) Patent Application Publication
(10) Pub. No.: US 2017/0296389 A1
(43) Pub. Date: Oct. 19, 2017

(54) Ear Plug Support

(71) Applicant: James W. Jahoda, Lebanon, CT (US)

(72) Inventor: James W. Jahoda, Lebanon, CT (US)

(21) Appl. No.: 15/478,376

(22) Filed: Apr. 4, 2017

Related U.S. Application Data
(60) Provisional application No. 62/317,704, filed on Apr. 4, 2016.

(51) Int. Cl. A61F 11/12; G02C 5/14; A61F 11/08; A42B 1/06; G02C 11/00; A42B 1/02

(52) U.S. Cl. CPC A61F 11/12; G02C 11/00; G02C 5/143; A42B 1/02; A42B 1/068; A42B 1/062; A61F 11/08

(57) ABSTRACT

An ear plug support article is provided and includes an ear plug, an ear plug arm having a first ear plug arm end and a second ear plug arm end, wherein the ear plug is connected to the first ear plug arm end and wherein the second ear plug arm end includes a mounting article and an ear plug arm support, wherein the ear plug arm support is configured to attach to a brim of a hat, wherein the ear plug arm support defines an arm support cavity configured to movably and securely contain the mounting article such that the ear plug arm is movable.

### CROSS REFERENCE TO RELATED APPLICATIONS

This application is related to and claims the benefit of the filing date of U.S. Provisional Patent Application Ser. No. 62/317,704 filed Apr. 4, 2017, the contents of which are incorporated herein by reference in its entirety.

### FIELD OF THE INVENTION

The present invention relates generally to ear plugs and more particularly to an ear plug support which is mounted to a wearable structure such as a hat or the arms of eye glasses.

### BACKGROUND OF THE INVENTION

Hearing loss caused by exposure to excessive noise is a growing problem among youth and other people that are in environments having loud events. The intensity of sound is typically measured in decibels, where on the decibel scale an increase in sound of 10 decibels means that the sound has increase in intensity by a factor of 10. It is well known that repeated and/or long exposure to noise at or above 85 decibels can cause hearing loss. And in fact there is a direct correlation between the length of exposure and the intensity (loudness) of the noise. The problem of hearing loss does not typically become an issue with most of the everyday sounds we hear because they are well below the 85 decibel threshold. For example, the sound of a standard refrigerator is typically around 45 decibels, while sound from a normal conversation is typically around 65 decibels. These are sound levels that are acceptable in our everyday lives. Unfortunately, not every sound we are exposed to is within the acceptable ranges. For example, a motorcycle (from everyday traffic), music (from a loud stereo or live concert) or a gunshot (from a shooting range) emits sounds that are typically in the range of 120 decibels to 150 decibels (or more).

One well known way to address at least some of these problems involves wearing ear protection, such as ear plugs, where some ear plugs are disposable and some are reusable. In the situation where the ear plugs are reusable, one problem that exists involves not losing the ear plugs or not dropping the ear plugs on the ground so that they get dirty and transmit infection to the wearer. This is typically addressed by tethering the ear plugs to a device that attaches to an article worn by the wearer, such as a pair of sunglasses. Unfortunately, the current methods for tethering ear plugs to a pair of sunglasses only includes attaching the ear plugs so that they hang down around the neck of the wearer. This is undesirable because while hanging down, the ear plugs can get caught on clothing and/or other articles and thus, become damaged or dirty.

### SUMMARY OF THE INVENTION

An ear plug support article is provided and includes an ear plug, an ear plug arm having a first ear plug arm end and a second ear plug arm end, wherein the ear plug is connected to the first ear plug arm end and wherein the second ear plug arm end includes a mounting article and an ear plug arm support, wherein the ear plug arm support is configured to attach to an arm of a pair of glasses, wherein the ear plug arm support defines an arm support cavity configured to movably and securely contain the mounting article.

An ear plug support article is provided and includes an ear plug, an ear plug arm having a first ear plug arm end and a second ear plug arm end, wherein the ear plug is connected to the first ear plug arm end and wherein the second ear plug arm end includes a mounting article and an ear plug arm support, wherein the ear plug arm support is configured to attach to a brim of a hat, wherein the ear plug arm support defines an arm support cavity configured to movably and securely contain the mounting article such that the ear plug arm is movable.

### BRIEF DESCRIPTION OF THE DRAWINGS

The foregoing and other features and advantages of the present invention will be more fully understood from the following detailed description of illustrative embodiments, taken in conjunction with the accompanying drawings in which like elements are numbered alike in the several Figures:

FIG. 1 is side view of the ear plug support associated with an arm of a pair of eye glasses, in accordance with one embodiment of the present invention.

FIG. 2 is a top down view of the ear plug support of FIG. 1.

FIG. 3 is a side view of the eye glass arm mount of the ear plug support of FIG. 1.

FIG. 4 is a top down view of the ear plug support of FIG. 1.

FIG. 5 is a side view of an ear plug support associated with a hat, in accordance with still yet another embodiment of the invention.

FIG. 6 is a side view of an ear plug support associated with a baseball cap, in accordance with still yet another embodiment of the invention.

FIG. 7 is a side view of an ear plug support, in accordance with another embodiment of the invention.

FIG. 8 is a side view of an ear plug support having a retractable ear plug, in accordance with another embodiment of the invention.

FIG. 9A is a side view of an ear plug arm support, in accordance with another embodiment of the invention.

FIG. 9B is a side view of the ear plug arm support of FIG. 9A, being associated with an ear plug arm of FIG. 1 and FIG. 8 and a hat, in accordance with another embodiment of the invention.

FIG. 9C is a side view of an ear plug arm, in accordance with another embodiment of the invention.

FIG. 10A is a side view of an ear plug arm support, in accordance with still yet another embodiment of the invention.

FIG. 10B is a side view of the ear plug arm support of FIG. 10A, being associated with an ear plug arm of FIG. 1, FIG. 8 and FIG. 9C and a hat, in accordance with still yet another embodiment of the invention.

FIG. 10C is a side view of the ear plug arm support of FIG. 10A associated with the brim of a hat.

### DETAILED DESCRIPTION OF THE INVENTION

In accordance with the present invention an ear plug support article is provided and may be constructed from one or more parts and pieces and may be constructed from any material suitable to the desired end purpose, such as plastic, composite, wood, metal, fiberglass material or any combination thereof.

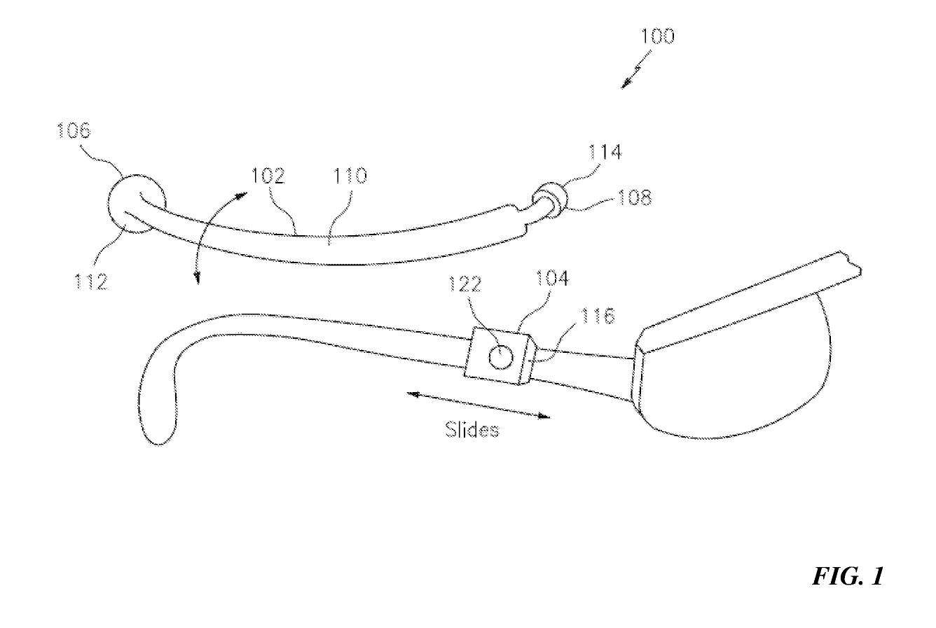

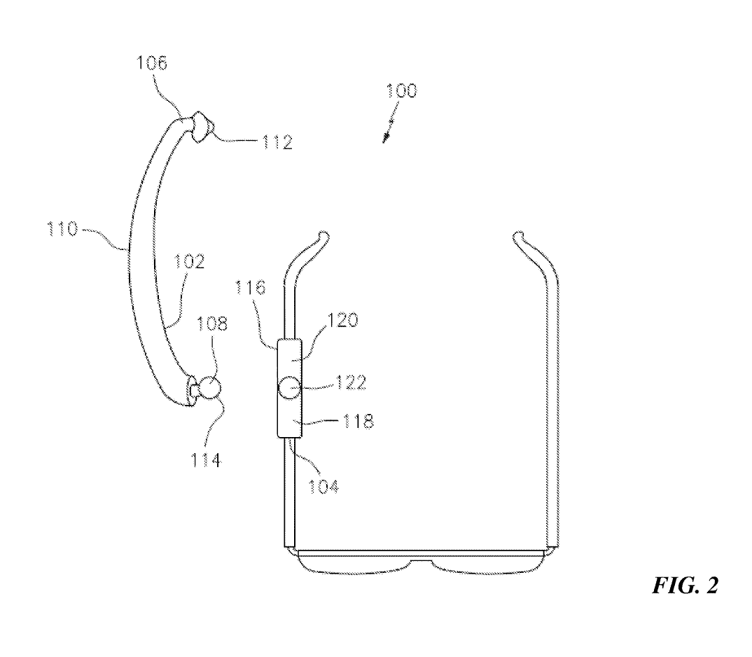

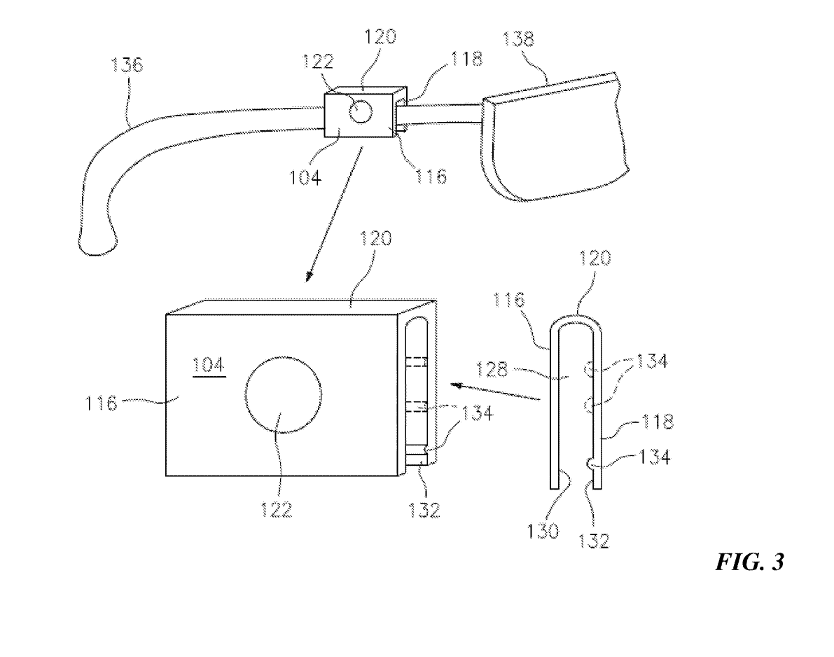

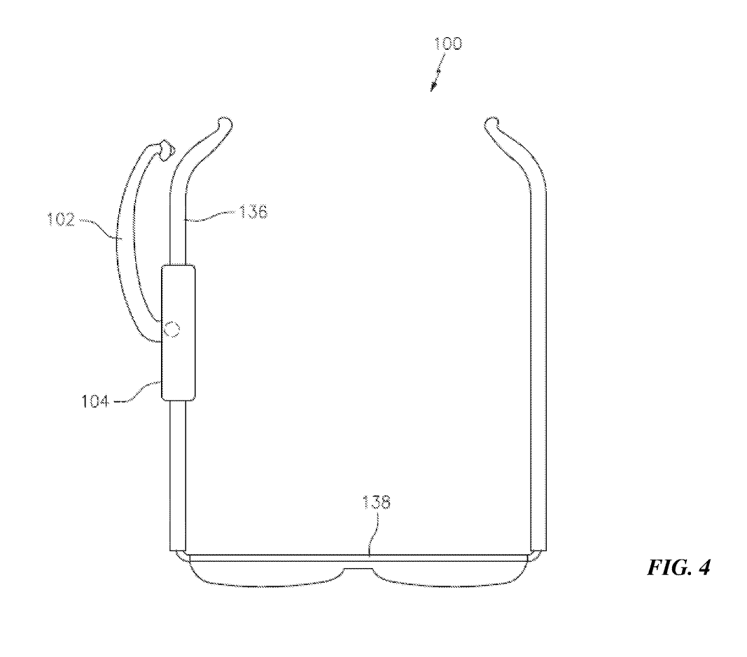

Referring to FIG. 1, FIG. 2, FIG. 3 and FIG. 4, an ear plug support article 100 is shown and includes an ear plug arm 102 and an ear plug arm support 104. The ear plug arm 102 includes a first ear plug arm end 106 and a second ear plug arm end 108, wherein the first ear plug arm end 106 is connected to the second ear plug arm end 108 via an ear plug arm middle portion 110. The first ear plug arm end 106 is configured to include an ear plug 112 and the second ear plug arm end 108 is configured to include a mounting article or ball 114. It is contemplated that the ear plug arm middle portion 110 may be rigid or may be malleable, as desired. The ear plug arm support 104 includes an arm support outer structure 116 connected to an arm support inner structure 118 via an arm support top 120. The arm support outer structure 116 defines a socket cavity 122 having a socket cavity surface 124, wherein the socket cavity 122 is sized and shaped to contain the mounting ball 114. It is contemplated that the socket cavity surface 124 may include one or more protrusions 126 configured to movably retain the mounting ball 114 within the socket cavity 122 when the mounting ball 114 and socket cavity 122 are associated with each other.

It should be appreciated that the arm support outer structure 116 and the arm support inner structure 118 are disposed to be substantially parallel with each other and connected together on one end by the arm support top 120 to define an arm support cavity 128, wherein the arm support cavity 128 is sized to snugly and movably contain a portion of the arm 136 of a pair of eye glasses 138. It should be further appreciated that the arm support outer structure 116 includes an arm support outer structure inner surface 130 and the arm support inner structure 118 includes an arm support inner structure inner surface 132. Additionally, the ear plug arm support 104 includes one or more ridges 134 (and/or protrusions) located on at least one of the arm support outer structure inner surface 130 and the arm support inner structure inner surface 132.

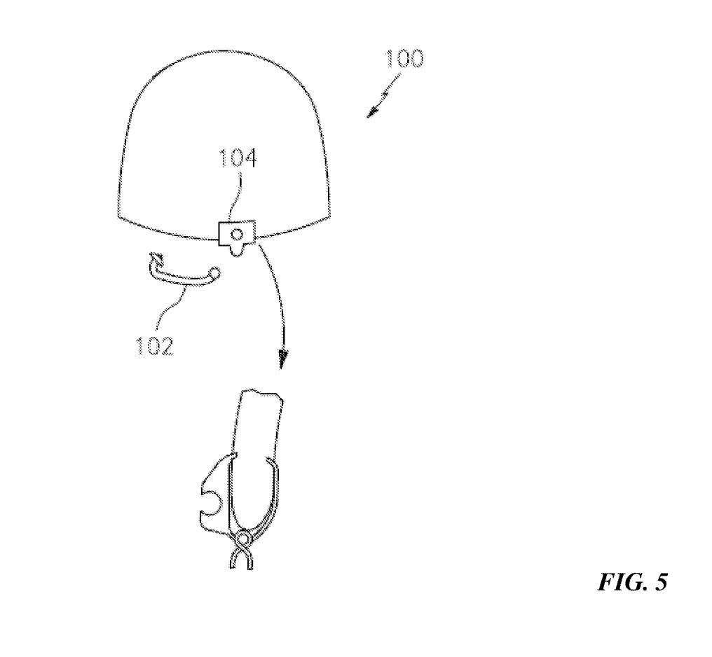

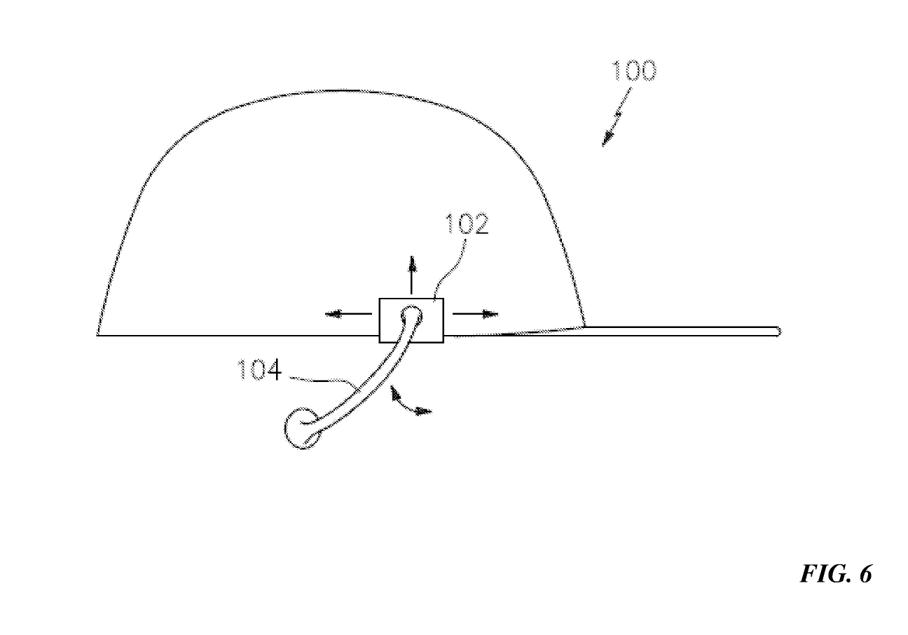

Referring again to the FIG.'s, the ear plug arm support 104 is associated with an arm 136 of a pair of eye glasses 138 such that the eye glass arm 136 is snugly and movably located within the arm support cavity 128. The ear plug arm 102 is associated with the ear plug arm support 104 such that the mounting ball 114 is snugly and movably located and contained within the socket cavity 122. This arrangement allows the ear plug arm 102 to rotate within the arm support cavity 128 and for the ear plug arm support 104 to move along the arm 136 of the eye glasses 138. This advantageously allows a user to adjust the ear plug support 100 so that the ear plugs can be used comfortably and removed as desired. Referring to FIG. 5 and FIG. 6, it should be appreciated that the ear plug support 100 may also be used to clip or attach to the brim of a hat.

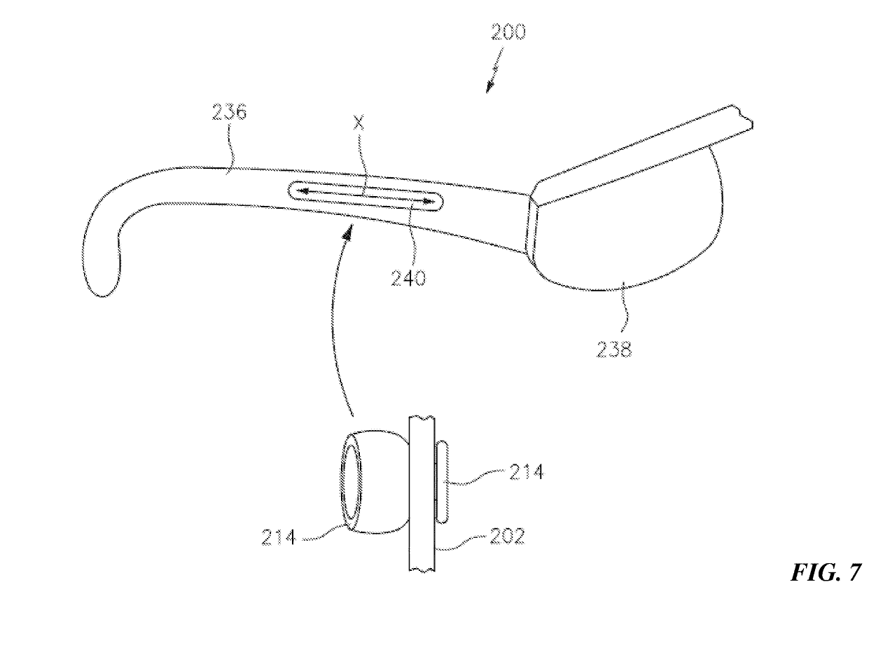

Referring to FIG. 7, an ear plug support 200 is shown in accordance with another embodiment of the invention. As shown, a pair of eye glasses 238 having eye glass arms 236 is shown where the eye glass arms 236 define a mounting slot 240 which extends partially along the length X of the eye glass arms 236. As with the previous embodiment, the ear plug support 200 includes an ear plug arm 202. The ear plug arm 202 includes a first ear plug arm end and a second ear plug arm end 208, wherein the first ear plug arm end is connected to the second ear plug arm end 208 via an ear plug arm middle portion. The first ear plug arm end is configured to include an ear plug and the second ear plug arm end 208 includes a mounting structure 214, wherein the mounting structure 214 is configured to movably and securely be contained within the mounting slot 240 to allow the ear plug arm 202 to rotate about an axis and move along a portion of the length of the eye glass arms 236.

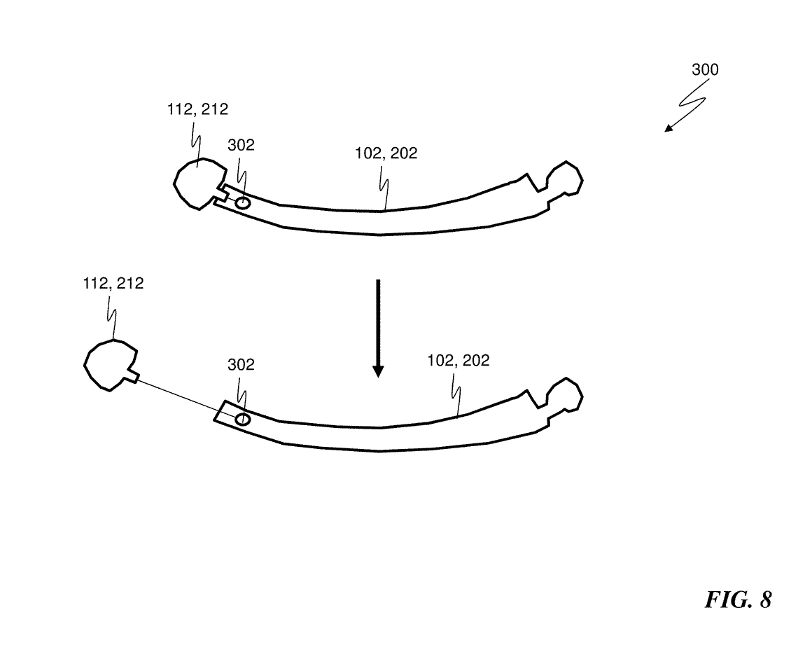

Referring to FIG. 8, it is contemplated that in another embodiment 300, the ear plug 112, 212 is movably and/or extendably associated with the ear plug arm 102, 202 via a resilient tethering device 302. This advantageously allows the ear plug 112, 212 to extend away from and back retract into the ear plug arm 102, 202. It should be appreciated that the tethering device 302 may be internal or external to the ear plug arm 102, 202.

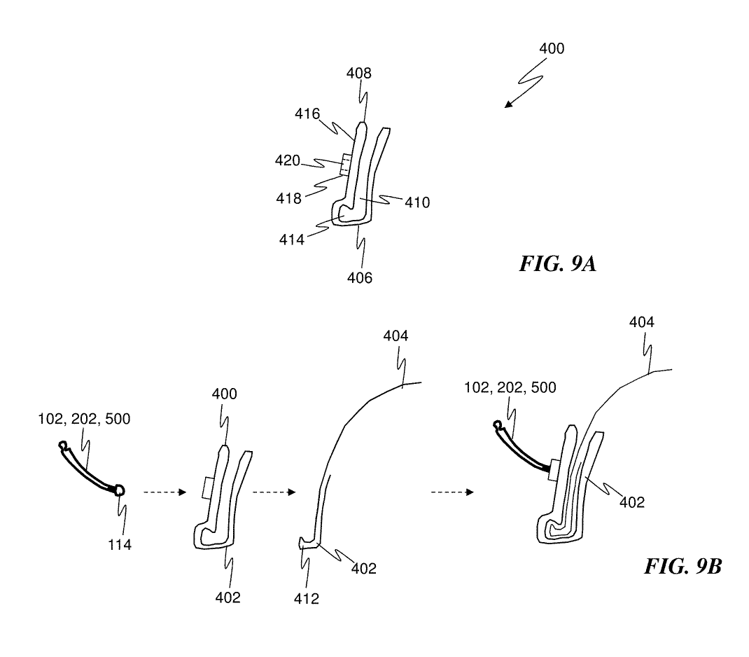

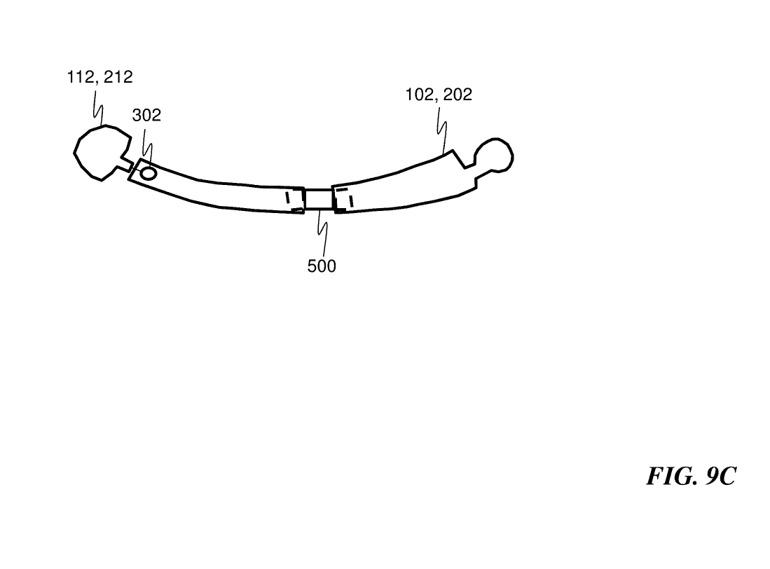

Referring to FIG. 9A and FIG. 9B, an ear plug arm support 400 is provided in accordance with another embodiment, wherein the ear plug arm support 400 is configured to connect to the brim 402 of a hat 404 (such as a hard hat i.e. construction hat or other type of hat). The ear plug arm support 400 includes a support structure base 406 and a support structure arm 408, wherein the support structure base 406 and the support structure arm 408 define a support structure cavity 410 which may be shaped to contain the brim 402 of the hat (or other type of hat) 404. In one embodiment, the brim 402 of the hat 404 may include a brim lip 412 and the support structure cavity 410 may include a brim lip portion 414 for containing the brim lip 412. The support structure base 406 and/or the support structure arm 408 may be shaped to fit with the contour of the surface of the hat 404. Additionally, the support structure arm 408 includes an outer surface 416 and an arm support outer structure 418 associated with the outer surface 416, where the arm support outer structure 418 defines a socket cavity 420 sized and shaped to contain the mounting ball 114 of the ear plug arm 102, 202 such that the mounting ball 114 is movably contained within the socket cavity 420 when the mounting ball 114 and socket cavity 420 are associated with each other.

Moreover, the support structure base 406 and the support structure arm 408 may be configured such that at least one of the support structure base 406 and the support structure arm 408 is movable relative to the other. This advantageously allows the brim 402 of the hat 404 to be inserted into and contained within the support structure cavity 410 to attach the ear plug arm support 400 to the hat 418. As such, when a user wears the hat 404 with the ear plug arm support 400 attached and the ear plug arm 102, 202 is associated with the arm support outer structure 418, the ear plug 112 can be inserted into the ear of the user. It should be appreciated that the ear plug arm support 400 may be located anywhere along the brim 402 of the hat 404 so that it can be adjusted to the comfort and usability of varying users. It should be appreciated that in at least one embodiment, the ear plug arm 102, 202 maybe adjustable in length. As one example and referring to FIG. 9C, the ear plug arm 102, 202 may include a telescoping structure 500 that allows the length of the ear plug arm 102, 202 to be adjustable in length as desired.

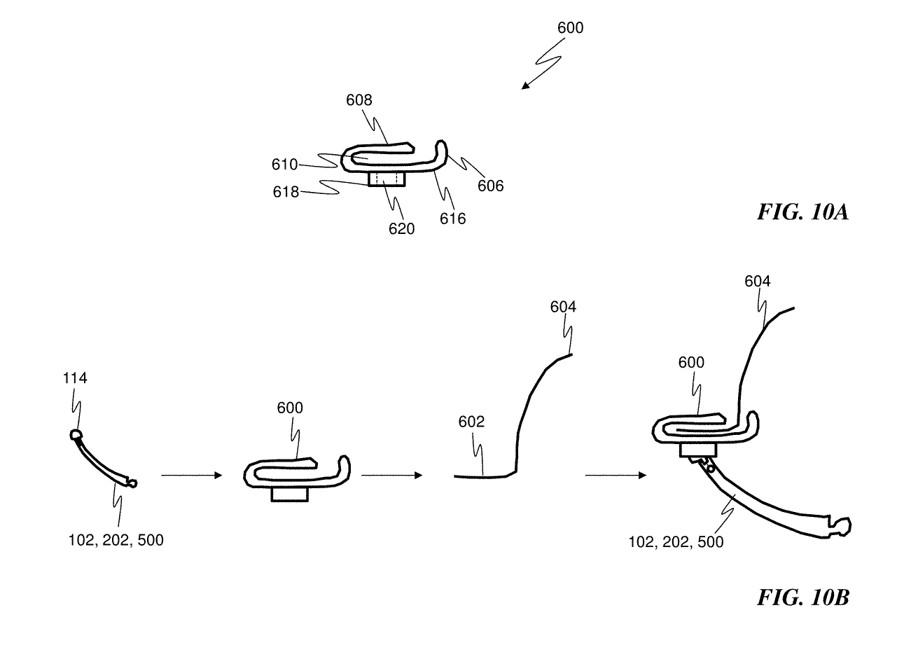

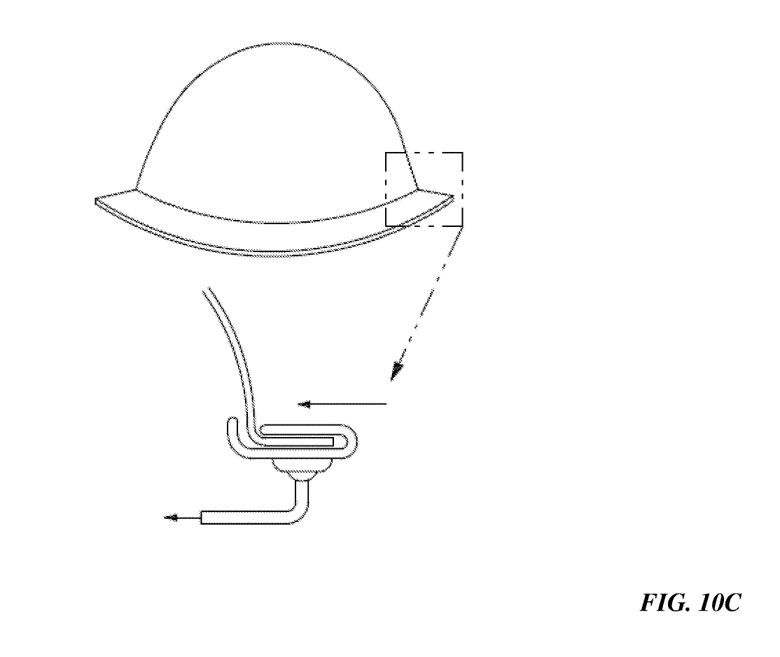

Referring to FIG. 10A, FIG. 10B and FIG. 10C, an ear plug arm support 600 is provided in accordance with yet another embodiment, wherein the ear plug arm support 600 is configured to connect to the brim 602 of a hat 604 (such as a hard hat i.e. construction hat or other type of hat). The ear plug arm support 600 includes a support structure base 606 and a support structure arm 608, wherein the support structure base 606 and the support structure arm 608 define a support structure cavity 610 which may be shaped to contain the brim 602 of the hat (or other type of hat) 604. The support structure base 606 and/or the support structure arm 608 may be shaped to fit with the contour of the surface of the hat 604. Additionally, the support structure arm 608 includes an outer surface 616 and an arm support outer structure 618 associated with the outer surface 616, where the arm support outer structure 618 defines a socket cavity 620, wherein the socket cavity 620 is sized and shaped to contain the mounting ball 114 of the ear plug arm 102, 202, 500 such that the mounting ball 114 is be movably contained within the socket cavity 620 when the mounting ball 114 and socket cavity 620 are associated with each other

As such, when a user wears the hat 604 with the ear plug arm support 600 attached and the ear plug arm 102, 202, 500 is associated with the arm support outer structure 618, the ear plug 112 can be inserted into the ear of the user. It should be appreciated that the ear plug arm support 600 may be located anywhere along the brim 602 of the hat 604 so that it can be adjusted to the comfort and usability of varying users.

It should be appreciated that while the invention has been described with reference to an exemplary embodiment, it will be understood by those skilled in the art that various changes, omissions and/or additions may be made and equivalents may be substituted for elements thereof without departing from the spirit and scope of the invention. Moreover, embodiments and/or elements of embodiments disclosed herein and within the scope of the invention may be combined as desired. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

What is claimed is:

1. An ear plug support article, comprising:
an ear plug;
an ear plug arm having a first ear plug arm end and a second ear plug arm end, wherein the ear plug is connected to the first ear plug arm end and wherein the second ear plug arm end includes a mounting article; and
an ear plug arm support, wherein the ear plug arm support is configured to attach to an arm of a pair of glasses, wherein the ear plug arm support defines an arm support cavity configured to movably and securely contain the mounting article.

2. The ear plug support article of claim 1, wherein the mounting article is a mounting ball.

3. The ear plug support article of claim 1, wherein when the ear plug arm is located within the arm support cavity, the ear plug arm rotates about an axis.

4. The ear plug support article of claim 1, wherein the ear plug arm support includes a support structure base and a support structure arm and wherein the ear plug arm support defines arm support cavity.

5. The ear plug support article of claim 4, wherein the arm support cavity is configured to moveably and securely contain the mounting article.

6. The ear plug support article of claim 1, wherein ear plug arm support defines a support structure cavity for containing and securely associating with a brim of a hat.

7. An ear plug support article, comprising:
an ear plug;
an ear plug arm having a first ear plug arm end and a second ear plug arm end, wherein the ear plug is connected to the first ear plug arm end and wherein the second ear plug arm end includes a mounting article; and
an ear plug arm support, wherein the ear plug arm support is configured to attach to a brim of a hat, wherein the ear plug arm support defines an arm support cavity configured to movably and securely contain the mounting article such that the ear plug arm is movable.

8. The ear plug support article of claim 7, wherein the mounting article is a mounting ball.

9. The ear plug support article of claim 7, wherein when the ear plug arm is located within the arm support cavity, the ear plug arm rotates about an axis.

10. The ear plug support article of claim 7, wherein the ear plug arm support includes a support structure base and a support structure arm and wherein the ear plug arm support defines arm support cavity.

11. The ear plug support article of claim 10, wherein the arm support cavity is configured to moveably and securely contain the mounting article.

12. The ear plug support article of claim 7, wherein ear plug arm support defines a support structure cavity for containing and securely associating with a brim of a hat.
